(19) United States
(12) Patent Application Publication
(10) Pub. No.: US 2014/0163405 A1
(43) Pub. Date: Jun. 12, 2014

(54) PHYSIOLOGICAL INFORMATION MEASUREMENT SYSTEM AND METHOD THEREOF

(71) Applicant: Indsutrial Technology Research Institute, Hsin-Chu (TW)

(72) Inventors: PANG-CHAN HUNG, New Taipei City (TW); KUAL-ZHENG LEE, Chiayi County (TW); LUO-WEI TSAI, Kaohsiung City (TW)

(21) Appl. No.: 13/922,578

(22) Filed: Jun. 20, 2013

(30) Foreign Application Priority Data
Dec. 11, 2012 (TW) 101146641

(51) Int. Cl. A61B 5/00; A61B 5/0255; A61B 5/08

(52) U.S. Cl. CPC A61B 5/72; A61B 5/0803; A61B 5/0255
USPC 600/508; 600/300; 600/529

(57) ABSTRACT

A physiological information measurement system includes at least one video capture unit, a calculating unit electrical coupled to the video capture unit and a display unit electrical coupled to the calculating unit. The video capture unit captures at least one video provided to the calculating unit. The calculating unit measures physiological information according to the video. The display unit shows the physiological information.

### CROSS REFERENCE TO RELATED APPLICATION

This application also claims priority to Taiwan Patent Application No. 101146641 filed in the Taiwan Patent Office on Dec. 11, 2012, the entire content of which is incorporated herein by reference.

### TECHNICAL FIELD

The present disclosure relates to a physiological information measurement system and method, and more particularly, to a system and a method for physiological information measurement.

### BACKGROUND

Heart rate, an index for cardiovascular disease, and respiratory rate, an index for sleep apnea, are important physiological information for human body. Medical personnel often determine physiological condition of patients according to the heart rate and the respiratory rate.

Conventional heart rate measuring equipments include pulse oximeter, sphygmomanometer and electrocardiograph. Conventional respiratory rate measuring equipments include spirometer, impedance pneumography and respiratory inductive plethysmography.

Measurement by the described equipments is almost contact-based, which often causes the patients discomfort. Besides, the equipments are expensive and seldom used by ordinary people.

To prevent the discomfort caused by the contact-based equipments, contact-free measuring equipments are therefore developed.

The contact-free measuring equipment utilizes single camera and single video region as a signal source which can correctly operate only for stable light sources and motionless objects (patient).

Even the patients are still, slight movement, facial expression change or improper camera shooting direction may influence the measurement and reduce correctness.

### SUMMARY

In an embodiment, the present disclosure provides a physiological information measurement system, including: at least one video capture unit, a calculating unit electrically coupled to the video capture unit, and a display unit electrically coupled to the calculating unit. The video capture unit captures at least one video data provided for the calculating unit to obtain physiological information displayed on the display unit.

In another exemplary embodiment, the present disclosure provides a physiological information measurement method, including the steps of: providing a plurality of video data, wherein each video data contains sequential image data, extracting and synchronizing the video data to obtain a synchronous features, transforming the features to independent components, detecting peak value of the independent components, selecting a representative component from the independent components to generate a physiological information, and displaying the physiological information.

Further scope of applicability of the present application will become more apparent from the detailed description given hereinafter. However, it should be understood that the detailed description and specific examples, while indicating exemplary embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The present disclosure will become more fully understood from the detailed description given herein below and the accompanying drawings which are given by way of illustration only, and thus are not limitative of the present disclosure and wherein:

FIG. 1 is a schematic view of a physiological information measurement system according to the present disclosure.

FIG. 2 is a flow chart of a physiological information measurement method according to the present disclosure.

FIG. 3 is a flow chart of peak detection method according to the present disclosure.

FIG. 4A is schematic view of a plurality of video capture units capturing video data according to the present disclosure.

FIG. 4B is schematic view of the video data according to the present disclosure.

FIG. 5A is schematic view of one video capture unit capturing video data according to the present disclosure.

FIG. 5B is schematic view of the video data according to the present disclosure.

FIG. 6 is a schematic view of capturing a plurality of respiratory rate diagrams in the present disclosure.

FIG. 7 depicts a plurality of feature diagrams in the present disclosure.

FIG. 8 depicts a plurality of synchronous feature diagrams according to the present disclosure.

FIG. 9 depicts a plurality of independent component diagrams according to the present disclosure.

### DETAILED DESCRIPTION

In the following detailed description, for purposes of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawing.

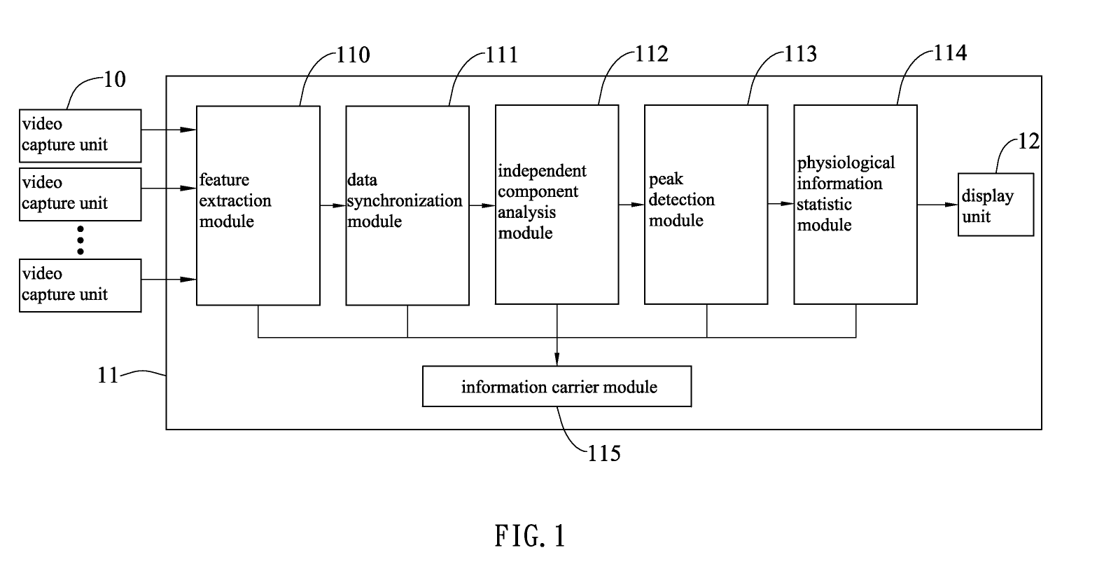

Please refer to FIG. 1, a physiological information measurement system of one embodiment of the present disclosure includes at least one video capture unit 10, a calculating unit 11 and a display unit 12. The video capture unit 10 can be a camera, a video file, a universal serial bus web camera (USB web camera), a camera for mobile devices, web information, web video streaming or field depth camera. The video capture unit 10 can be single one or plural.

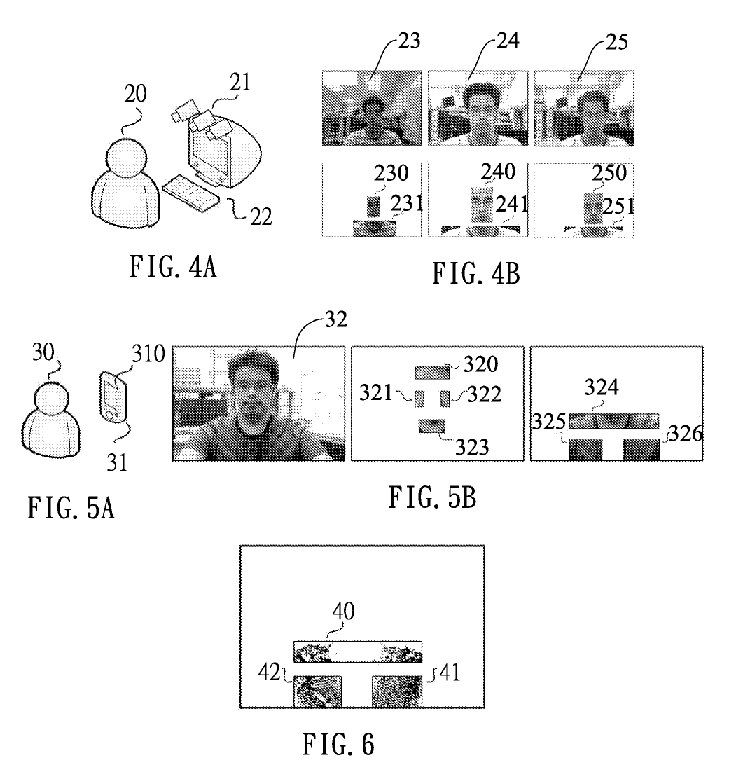

Please refer to FIG. 4A, which depicts an embodiment of a video capture unit 21 of the disclosure. The video capture unit 21 includes several USB web cameras. Each USB web camera captures video data of a person. As shown in FIG. 4B, the images include a first video 23, a second video 24 and a third video 25. The first video 23, the second video 24 and the third video 25 are displayed in a calculating unit 22.

Please refer to FIG. 5A, which depicts an embodiment of a video capture unit 310 of the disclosure. The video capture unit 310 is a camera for a mobile device 31. As shown in FIG. 5B, the video capture unit 310 captures at least one video 32 of a person 30. The video 32 is shown on the mobile device 31.

The calculating unit 11 is electrically coupled to the video capture unit 10. The calculating unit 11 includes a feature extraction module 110, a data synchronization module 111, an independent component analysis module 112, a peak detection module 113, a physiological information statistic module 114 and an information carrier module 115.

The feature extraction module 110 is electrically coupled to the video capture unit 10. The feature extraction module 110 receives video data from the video capture units 10 and generates a plurality of features.

Please refer to FIG. 4B again, the first image 23, the second image 24 and the third image 25 have regions 230, 231, 240, 241, 250 and 251 respectively. The regions 230, 231, 240, 241, 250 and 251 can be treated as the described video data. For example, regions 230, 240 and 250 can be used to measure a heart rate. The regions 231, 241 and 251 can be used to measure a respiratory rate. However, they are not limited to measure the heart rate and the respiratory rate.

The feature extraction module 110 utilizes a temporal differencing method to obtain motion pixels 40, 41 and 42 in the regions 324, 325 and 326 of FIG. 5B, which are treated as video data. As shown in FIG. 6, if the amount of the motion pixels 40, 41 and 42 is figured out, then the amount can be treated as features for the video data.

The data synchronization module 111 receives the features from the feature extraction module 110 and synchronizes the features.

The independent component analysis module 112 receives the synchronous features and generates a plurality of independent components.

The peak detection module 113 receives the independent components and generates peak information and several serial peak signals.

The physiological information statistic module 114 receives and analyzes the serial peak signals to select one of the independent components. The physiological information statistic module 114 generates a physiological signal based on the selected independent component.

The information carrier module 115 is informatively connected to the feature extraction module 110, the data synchronization module 111, the independent component analysis module 112, the peak detection module 113 and the physiological information statistic module 114. The information carrier module 115 can be an inner or outer data base or a fixed or mobile memory.

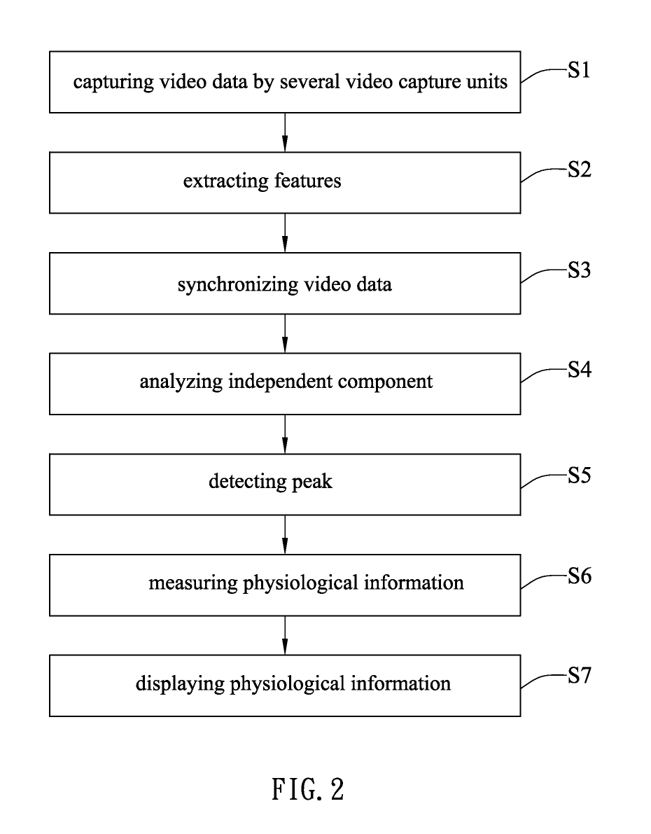

Please refer to FIG. 2, a physiological information measurement method of the present disclosure includes the steps of:

Step 1 (S1), providing K groups of video data, and each group of video dada includes sequential image data of physical physiological information regions. For example, the physical physiological information region can be a face region, a neck region, an arm region, a shoulder region, a chest-abdominal region, a left chest region or a right chest region.

The physiological information regions can be obtained by a face detecting process, a skin color detecting process or a manually figuring process. For example the face detecting process can refer to M.-Z. Poh, D. J. McDuff, and R. W. Picard, “Advancements in noncontact, multiparameter physiological measurements using a webcam,” IEEE Trans. Biomedical Engineering, vol. 58, pp. 7-11, January 2011. The skin color detecting process can refer to K.-Z. Lee, P.-C. Hung, and L.-W. Tsai, “Contact-free heart rate measurement using a camera,” in Proc. Ninth Conference on Computer and Robot Vision, 2012, pp. 147-152. The manually figuring process can refer to K. S. Tan, R. Saatchi, H. Elphick, and D. Burke, “Real-time vision based respiration monitoring system,” in Proc. International Symposium on Communication Systems Networks and Digital Signal Processing, 2010, pp. 770-774.

Referring to FIG. 1, the format of the video data is one of three primary color format (red, green and blue, RGB format), true-color space format (luminance, chrominance and chrome, YUV format) or color attribute format (hue, saturation and value, HSV format). The video data captured by the video capture unit 10 are saved in the information carrier module 115 based on time sequence for later access and calculation.

For example, the K groups of video data are obtained by shooting a person with the video capture units 10. The K groups of video data are provided to the calculating unit 11.

The K groups of video data can also be obtained by shooting a person with the video capture units 10 built in a mobile device such as a mobile phone.

As described above, I_{ff}^{k} is the image data, where k=1, 2, 3, . . . , K. I_{f}^{k} is the f^{th} frame in the k^{th} video. T(I_{f}^{k}) is the time for capturing image I_{f}^{k}. Unit of the time can be ms, μs, s, minute or hour.

S2, the feature extraction module 110 obtains features including physiological information from each image I_{f}^{k} to analyze physiological information.

For example, if the physiological information is a heart rate, then the heart rate is obtained by the average color of skin region accompany with a weighted statistical method. The weighted statistical method can refer to K.-Z. Lee, P.-C. Hung, and L.-W. Tsai, “Contact-free heart rate measurement using a camera,” in Proc. Ninth Conference on Computer and Robot Vision, 2012, pp. 147-152. Therefore, when heart rate is measured, the feature u_{f}^{k} of the f^{th} frame in the k^{th} video can be a weighting value for color average.

If the physiological information is a respiratory rate, then the respiratory rate is obtained by measuring the movement of chest. The movement is obtained by a temporal differencing method. The temporal differencing method can refer to K. S. Tan, R. Saatchi, H. Elphick, and D. Burke, “Real-time vision based respiration monitoring system,” in Proc. International Symposium on Communication Systems Networks and Digital Signal Processing, 2010, pp. 770-774. Therefore, when respiratory rate is measured, the feature u_{f}^{k} of the f^{th} frame in the k^{th} video can be an amount of motion pixels.

S3, since the frame rate of each video data is not static, frame rate is defined as the number of frames captured in a specific period. For example, the video capture units 10 has a frame rate N fames/sec, where N is a constant such as 10, 20, 30, 60, 120, 150, 180 or 300.

As described above, the time points of the video data is not synchronous due to unstable frame rate of each video data. A common frequency H fps is provided for each video data to obtain a synchronous feature νₜ^{k} at time t by interpolation method, where T(νₜ^{k})=1000×t/H is the time index of the synchronous feature νₜ^{k}, t=1, 2, 3, . . .

The synchronous feature vₜ^{k} of each video data has the same time index T(νₜ^{k}) at time t after synchronization.

If the feature u_{f}^{k} of a known image I_{f}^{k} has a time index T (I_{f}^{k}), the synchronous feature νₜ^{k} at time t can be obtained by an interpolation method. The interpolation method can be a linear interpolation method, a bilinear interpolation method or a bicubic interpolation method. These interpolation methods refer to J. G. Proakis and D. K. Manolakis, Digital Signal Processing (4th Edition): Prentice Hall, 2006.

For example, the synchronous features are obtained by a linear interpolation method, which is measured by the following equation:

$${v}_{t}^{k} = {u}_{f}^{k} + \frac{( {u}_{f + 1}^{k} - {u}_{f}^{k} ) \times ( T ( {v}_{t}^{k} ) - T ( {I}_{f}^{k} ) )}{T ( {I}_{f + 1}^{k} ) - T ( {I}_{f}^{k} )}$$

where T(I_{f}^{k})≦T(νₜ^{k})≦T(I_{f+1}^{k})
the synchronous features are obtained by a data synchronization module 111.

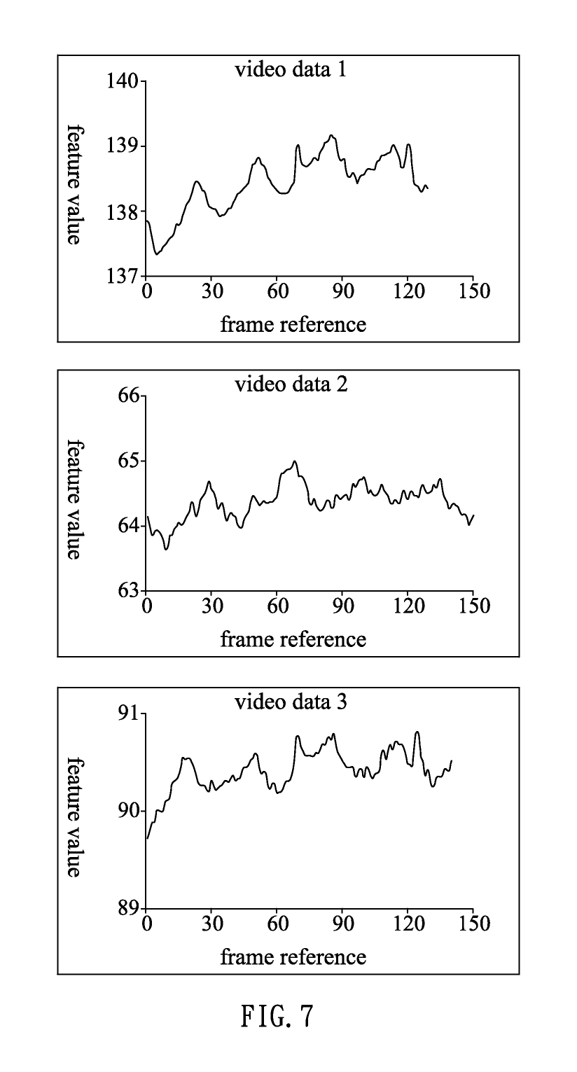

Please refer to FIG. 7, features of the regions 230, 240 and 250 in FIG. 4B are shown. The features can be a series of heart rate feature under the frame rate of each video capture unit is 30 fps and the measurement is performed for 5 seconds.

Supposed that the three video data have unstable frame rate, only 129 frames, 150 frames and 140 frames are captured. In addition, since each video capture unit has different characteristics, the captured features are different. Three average values of feature series are 138.43, 64.38 and 90.42 respectively.

A common frequency H fps is therefore defined and provided to each video data to obtain the synchronous feature νₜ^{k} at time t by the interpolation method.

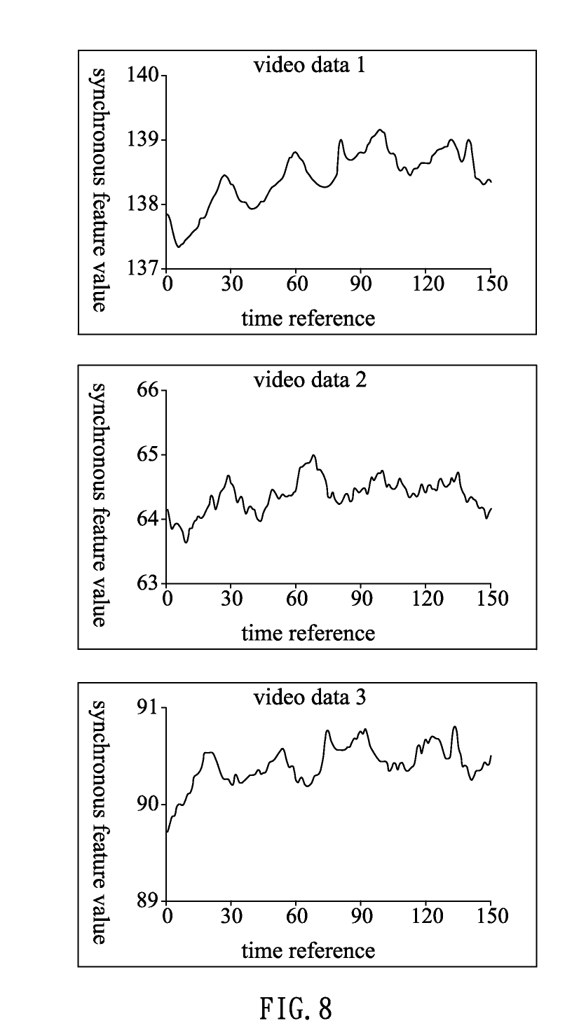

FIG. 8 shows the synchronous features for heart rate (after step S3). All features νₜ^{k} at time t of different groups have the same time index T(νₜ^{k}) .

S4, in addition to the physiological information, the video data also implicitly includes periodical variation of environment light (blinking lamp), periodical regulation of camera (automatic light compensation) and other variations caused by movement or facial expression change. If multiple groups of video data are measured simultaneously, since each video data includes the same physiological information, an independent component analysis method is utilized to extract stable signals from the video data. The independent component analysis method utilizes a linear transformation process to transform signals to a combination of non-Gaussian distributed signals which are statistically independent. The independent component analysis refers to A. Hyvärinen, J. Karhunen, and a. E. Oja, Independent Component Analysis. New York: John Wiley & Sons., 2001.

If N is the number of features which are intended to be analyzed. The value of N depends on the common frequency H fps and a reasonable value of the measured physiological information. For example, if N for the heart rate is defined as 5H, and N for the respiratory rate is defined as 30H, that means the heart rate and the respiratory rate use 5 seconds and 30 seconds as their input features respectively.

zₜ is a matrix of all features at time t

$${z}_{t} = {[ \begin{matrix}{v}_{t}^{1} & … & {v}_{t + N - 1}^{1} \\ ⋮ & ⋱ & ⋮ \\ {v}_{t}^{K} & … & {v}_{t + N - 1}^{K}\end{matrix} ]}_{K \times N}$$

zₜ is transformed to a matrix of statistically non-Gaussian independent components. zₜ=Axᵢ, where A is a mixing matrix. Since A and xᵢ is unknown, zₜ can be rewritten as

$${y}_{t} = {Wz}_{t} = {[ \begin{matrix}{y}_{t}^{1} & … & {y}_{t + N - 1}^{1} \\ ⋮ & ⋱ & ⋮ \\ {y}_{t}^{K} & … & {y}_{t + N - 1}^{K}\end{matrix} ]}_{K \times N}$$

where W is a demixing matrix similar to matrix A. If a demixing matrix W satisfies W≈A⁻¹, the independent component matrix yₜ≈xₜ, and yₜ^{k} is the value of the k^{th} independent component at time t.

The independent conponents are obtained by the independent component analysis module 112.

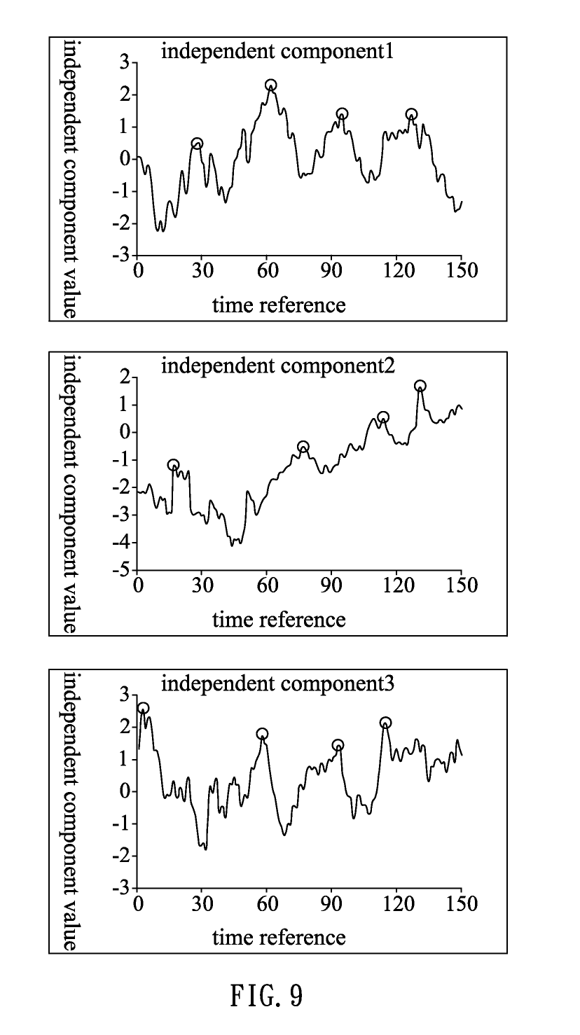

Referring to FIG. 9, the three independent components are analyzed, wherein N=5H and H=30 fps. As shown in FIG. 9, peaks are indicated by small circles. Each independent component has four peaks. The peak detection method is described in the step S5.

In step S5, the peak of the independent component yₜ is detected to obtain the signal period.

In the peak detection step, noise of signals is filtered out by a low pass filter or a median filter. Afterwards, local extreme values are searched to determine peaks' location. The signals are the described independent components. The peak detection method can refer to J. G. Proakis and D. K. Manolakis, Digital Signal Processing (4th Edition): Prentice Hall, 2006.

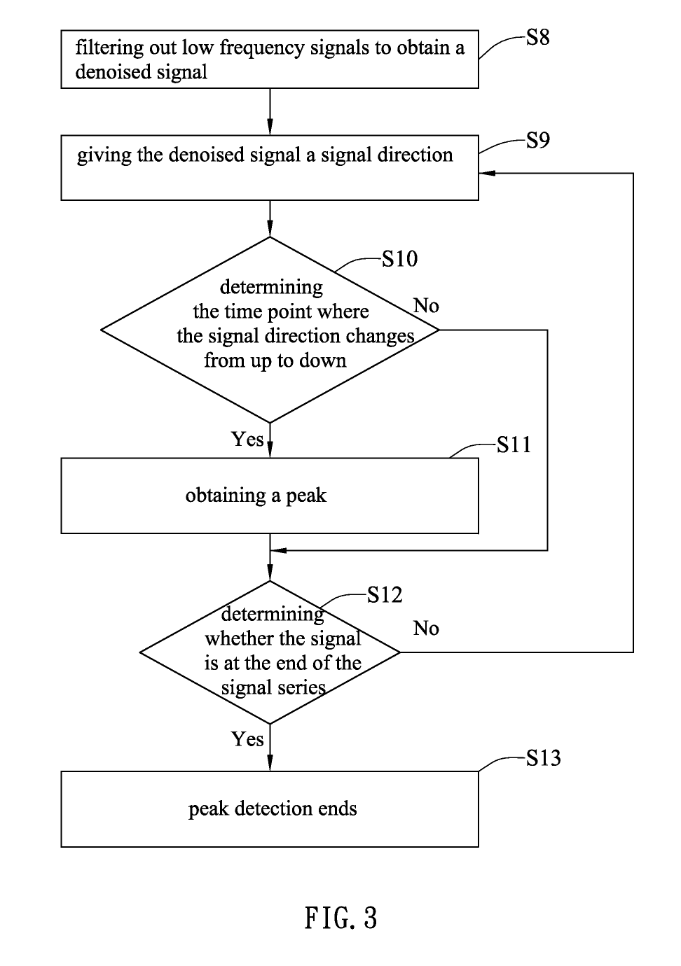

Referring to FIG. 3, peak detection method for each independent component is described as follows.

In step S8, low frequency signals of each independent component are filtered out by a filter to obtain a denoised signal matrix oₜ, where oₜ^{k} is the value of the k^{th} group of denoised signal at time t.

In step S9, each denoised signal oₜ^{k} is given a corresponding signal direction Dₜ^{k} which can be up, down or none.

Dₜ^{k} is given an initial value which is none, i.e. Dₜ^{k}=NONE.

When oₜ^{k}−oₜ₋₁^{k}>0, the signal direction is up, and when oₜ^{k}−oₜ₋₁^{k}<0, the signal direction is down. The signal direction Dₜ^{k} is therefore determined

In step 510, determine whether the signal direction changes from up to down at current time t. If the k^{th} group of the denoised signal has down direction at time t, and has up direction at time t−1, i.e. Dₜ^{k}=DOWN, Dₜ₋₁^{k}=UP, then a time point pᵢ^{k} is obtained (S11), where pᵢ^{k} is the time point of the i^{th} peak of the k^{th} group of the denoised signals oₜ^{k}, i=1, 2, 3, . . . nₖ, nₖ is the peak number of the k^{th} group of the denoised signals.

In step S12, if the time t is not the point where the signal direction changes from UP to DOWN or a new peak is obtained, then determine whether the signal is the last one of the signal series. If the signal is the last one of the signal series, the peak detection ends (S13); if the signal is not the last one, then return to step S9 to determine the signal direction of next time point.

The peak detection is performed by the peak detection module 113.

In step S6, peak-peak interval (PPI) between two adjacent peaks is calculated and analyzed to select a stable independent component to be the representative component.

The qⱼ^{k} represents the j^{th} PPI of the k^{th} group of the independent components, where j=1, 2, 3, . . . , nₖ−1. the value of qⱼ^{k} is obtained by the following equation:

$${q}_{j}^{k} = \{ \begin{matrix}\vert {p}_{j + 1}^{k} - {p}_{j}^{k} \vert & if {n}_{k} \geq 2 , \\ N & else .\end{matrix}$$

The Sₖ represents the variance of the PPI of the k^{th} group of the independent components. The independent component with the minimal variance (the most stable one) is selected as the representative component. The variance of PPI is calculated by the following equation:

$${S}_{k} = \{ \begin{matrix}\frac{1}{{n}_{k} - 1} {\sum }_{j = 1}^{{n}_{k} - 1} {( \overline{{q}^{k}} - {q}_{j}^{k} )}^{2} & if {n}_{k} \geq 2 , \\ {N}^{2} & else .\end{matrix}$$

The q̅^̅{̅k̅}̅ represents average of PPI of the k^{th} group of the independent components. The average is calculated by the following equation:

$$\overline{{q}^{k}} = \frac{1}{{n}_{k} - 1} {\sum }_{j = 1}^{{n}_{k} - 1} {q}_{j}^{k}$$

The average q̅^̅{̅k̅}̅ is selected to calculate the physiological information value R. R is calculated by the following equation:

$$R = \frac{60 \times H}{\overline{{q}^{k}}}$$

The independent component with the minimal variance is selected as the representative component. The average PPI q̅^̅{̅k̅}̅ of the representative component can be utilized to obtain the physiological information.

The physiological information is obtained by the physiological information statistic module 114.

In step S7, the physiological information obtained in step S6 is displayed on the display unit 12.

Information or data obtained by the feature extraction module 110, the data synchronization module 111, the independent component analysis module 112, the peak detection module 113 and the physiological information statistic module 114 can be saved in the information carrier module 115 or loaded from the information carrier module 115.

In the present disclosure, several video capture units are utilized to capture several video data. The video capture units can be various kinds of camera or information from internet.

In the present disclosure, measurement of the physiological information is automatic and noncontact based, which can reduce uncomfortable feeling. Besides, the influence caused by unstable signal is also reduced.

With respect to the above description then, it is to be realized that the optimum dimensional relationships for the parts of the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present disclosure.

What is claimed is:

1. A physiological information measurement system, comprising:
at least one video capture unit;
a calculating unit electrically coupled to the video capture unit; and
a display unit electrically coupled to the calculating unit,
wherein the video capture unit captures at least one video data provided for the calculating unit to obtain a physiological information displayed on the display unit.

2. The physiological information measurement system as claimed in claim 1, wherein the video capture unit is one of a camera, a video file, a USB webcam, a camera for mobile devices, a web information, a video streaming or a 3D depth camera.

3. The physiological information measurement system as claimed in claim 1, wherein the calculating unit comprises:
a feature extraction module electrically coupled to video capture unit to receive the video data and generate a plurality of features;
a data synchronization module receiving and synchronizing the features;
an independent component analysis module receiving the synchronous features and generating a plurality of independent components;
a peak detection module receiving the independent components and generating a plurality of serial peak signals;
a physiological information statistic module receiving the serial peak signals, selecting an independent component from the serial peak signals and generating a physiological information according to the independent component; and
an information carrier module carrying the physiological information.

4. The physiological information measurement system as claimed in claim 3, wherein the information carrier module comprises a data base or a memory.

5. A physiological information measurement method, comprising the steps of:
providing a plurality of video data, wherein each video data has sequential image data;
extracting and synchronizing the video data to obtain synchronous features;
converting the features to independent components;
detecting peak values of the independent components;
selecting a representative component from the independent components to generate a physiological information; and
displaying the physiological information.

6. The physiological information measurement method as claimed in claim 5, wherein the sequential image data comprise a physical physiological information region.

7. The physiological information measurement method as claimed in claim 6, wherein the physical physiological information region is a face region, a neck region, an arm region, a shoulder region, a chest-abdominal region, a left chest region or a right chest region.

8. The physiological information measurement method as claimed in claim 7, wherein the physical physiological information region is obtained by a face detecting process, a skin color detecting process or a manually figuring process.

9. The physiological information measurement method as claimed in claim 5, wherein the format of the video data is a three primary colors format, a true-color format or a color attribute format.

10. The physiological information measurement method as claimed in claim 5, wherein the physiological information comprises a heart rate or a respiratory rate.

11. The physiological information measurement method as claimed in claim 10, wherein the heart rate is obtained by an average color of the images and a weighted average method.

12. The physiological information measurement method as claimed in claim 10, wherein the respiratory rate is obtained by temporal differencing of the images.

13. The physiological information measurement method as claimed in claim 5, further comprising:
providing a common frequency and an interpolation method to obtain the synchronous features from the video data.

14. The physiological information measurement method as claimed in claim 13, wherein the interpolation method is a linear interpolation method, a bilinear interpolation method or a bicubic interpolation method.

15. The physiological information measurement method as claimed in claim 5, wherein the features are transformed to a combination of non-Gaussian distributed signals which are statistically independent by a linear transformation method.

16. The physiological information measurement method as claimed in claim 5, wherein in the peak detection step, noises of the independent components are filtered by a low pass filter or a median filter first. Then, the local extreme values of the independent components are searched to determine peaks' location.

17. The physiological information measurement method as claimed in claim 16, wherein the peak detection step comprising the steps of:
filtering out low frequency signals of each independent component to obtain a plurality of denoised signal traces;
providing corresponding signal direction, which is up, down or none, for each denoised signals;
setting an initial value to specify the signal direction to be none;
when oₜ^{k}−Oₜ₋₁^{k}>0, the signal has an up direction, and when oₜ^{k}−oₜ₋₁^{k}<0, the signal has a down direction, where oₜ^{k} is a value of a denoised signal at time t;
determining the time t on which the signal direction changes from up to down, if the denoised signal has a down direction at a time point and has an up direction at the previous time point, then a peak is obtained; and
determining whether a signal is the last signal when the signal direction of the signal is not down, if the signal is the last signal, then the peak detection step ends, and if the signal is not the last signal, then determine the signal direction.

18. The physiological information measurement method as claimed in claim 5, wherein the representative component is selected according to variances of peak-peak intervals, and the independent component having the minimal variance is selected as the representative component.

19. The physiological information measurement method as claimed in claim 18, wherein an average value of the peak-peak intervals is calculated from the representative component to obtain the physiological information.

20. The physiological information measurement method as claimed in claim 5, wherein the video data are caught by one video capture unit or a plurality of video capture units.
